(19) United States
(12) Patent Application Publication
(10) Pub. No.: US 2011/0123981 A1
(43) Pub. Date: May 26, 2011

(54) FTO GENE POLYMORPHISMS ASSOCIATED TO OBESITY AND/OR TYPE II DIABETES

(75) Inventors: Christian Rafael Dina, Paris (FR); Sophie Catherine Gallina Delamare, Hellemmes (Lille) (FR); Jean-Claude Chevre, Lille (FR); David Jean-Claude Meyre, Marpent (FR); Philippe Froguel, Bagnolet (FR)

(73) Assignees: CENTRE NATIONAL DE LA RECHERCHE SCIENTIFIQUE (CNRS; UNIVERSITE DE DROIT ET DE LA SANTE DE LILLE 2; INSTITUT PASTEUR DE LILLE

(21) Appl. No.: 12/594,319

(22) PCT Filed: Apr. 3, 2008
(86) PCT No.: PCT/EP2008/054031

§ 371 Date: Oct. 1, 2009

Related U.S. Application Data
(60) Provisional application No. 60/909,826, filed on Apr. 3, 2007.

(30) Foreign Application Priority Data
May 25, 2007 (EP) 07108984.1

(51) Int. Cl. C12Q 1/68

(52) U.S. Cl. 435/6

(57) ABSTRACT

The present invention provides means and methods for risk assessment and/or diagnosis and/or prognosis of obesity and/or type II diabetes in humans, based on the detection of nucleic acid biomarkers belonging to, or associated with, a set of SNPs in the fatso (FTO) gene. The present invention also provides means and methods for identifying a SNP haplotype associated with obesity and/or type II diabetes susceptibility in humans, for selecting pharmaceutical agents useful in prevention and/or treatment of obesity and/or type II diabetes in humans, for haplotyping the fatso (FTO) gene in humans.

The present invention relates to means for diagnosing, prognosing, treating and/or preventing obesity and/or type II diabetes in humans.

More precisely, the present invention provides means and methods for risk assessment and/or diagnosis and/or prognosis of obesity and/or type II diabetes in humans, based on the detection of nucleic acid biomarkers belonging to, or associated with, a set of SNPs (for “single nucleotide polymorphisms”) in the fatso (FTO) gene.

The present invention also provides means and methods for identifying a SNP haplotype associated with obesity and/or type II diabetes susceptibility in humans, as well as for selecting pharmaceutical agents useful in prevention and/or treatment of obesity and/or type II diabetes in humans.

Obesity is a condition in which the natural energy reserve, stored in the fatty tissue of humans and other mammals, is increased to a point where it is associated with certain health conditions or increased mortality.

Obesity is both an individual clinical condition and is increasingly viewed as a serious public health problem. Excessive body weight is now commonly known to predispose to various diseases, particularly cardiovascular diseases, sleep apnea, osteoarthritis, and diabetes (mellitus) type II. More precisely, obesity, especially central obesity (male-type or waist-predominant obesity), is an important risk factor for the “metabolic syndrome” (“syndrome X”), the clustering of a number of diseases and risk factors that heavily predispose for cardiovascular diseases. These risk factors are diabetes (mellitus) type II, high blood pressure, high blood cholesterol, and triglyceride levels (combined hyperlipidemia). An inflammatory state is present, which—together with the above—has been implicated in the high prevalence of atherosclerosis, and a prothrombotic state may further worsen cardiovascular risk.

In the clinical setting, obesity is typically evaluated by measuring BMI (for “body mass index”), waist circumference, and evaluating the presence of risk factors and comorbidities. In epidemiological studies, BMI alone is used as an indicator of prevalence and incidence of obesity. BMI is calculated by dividing the subject's weight in kilograms by the square of his/her height in metres:

BMI=(kg/m²) or BMI=[weight(lbs.)×703/height(inches)²]

Generally, it is considered that:
a BMI less than 18.5 is underweight
a BMI of 18.5-24.9 is normal weight
a BMI of 25.0-29.9 is overweight
a BMI of 30.0-39.9 is obese
a BMI of 40.0 or higher is severely (or morbidly) obese
also, a BMI of 35.0 or higher in the presence of at least one other significant comorbidity is usually classified as morbid obesity.

Factors that have been suggested to contribute to the development of obesity include, not only overeating, but also:
genetic factors and some genetic disorders (e.g., Prader-Willi syndrome);
underlying illness (e.g., hypothyroidism);
certain medications (e.g., atypical antipsychotics);
sedentary lifestyle; etc.

Obesity is often given to result from a combination of genetic and non-genetic factors. In this respect, the causative gene(s) is(are) still to be identified.

Today, obesity is seen as the biggest health problem facing developed and emerging countries.

Among all the means that have been made available for combating obesity, bariatric surgery is being increasingly used. This technique consists of placing a silicone ring around the top of the stomach to help restrict the amount of food eaten in a sitting. Other more invasive surgery techniques, that cut into or reroute any of the digestive tract, have been also used. However, all of these surgeries comme with risk to the patient and they do not guarantee either successful weight loss or reduced morbidity and mortality.

As a consequence, there is a need in the art for new drugs that would be really efficient for combating obesity. In this regard, identifying the gene(s) that is(are) involved in obesity onset, and thus that is(are) promising candidate therapeutic target(s), is one of the more crucial concerns of scientists and medical staffs.

This is precisely this need that the present invention aims at satisfying by disclosing the most significant association reported so far between a genetic factor and obesity. Indeed, the present invention is based on the finding that several SNPs (for “single nucleotide polymorphisms”) in fatso (FTO) locus are highly and significantly associated with early onset and severe obesity, as well as with the obesity related type II diabetes, in European population.

SNPs represent one of the most common forms of genetic variation. These polymorphisms appear when a single nucleotide in the genome is altered (such as via substitution, addition or deletion). Each version of the sequence with respect to the polymorphic site is referred to as an “allele” of the polymorphic site. SNPs tend to be evolutionary stable from generation to generation and, as such, can be used to study specific genetic abnormalities throughout a population. If SNPs occur in the protein coding region, it can lead to the expression of a variant, sometimes defective, form of the protein that may lead to the development of a genetic disease. Some SNPs may occur in non-coding regions, but nevertheless, may result in differential or defective splicing, or altered protein expression levels. SNPs can therefore serve as effective indicators of a genetic disease. SNPs can also be used as diagnostic tools for identifying individuals with a predisposition for a disease, genotyping the individual suffering from the disease, and facilitating drug development based on the insight revealed regarding the role of target proteins in the pathogenesis process.

For the avoidance of doubt, the methods of the invention do not involve diagnosis practised on the human body. The methods of the invention are preferably conducted on a sample that has previously been removed from the individual. The kits of the invention, described hereunder, may include means for extracting the sample from the individual.

The methods of the invention allow the accurate evaluation of risk for an individual's health due to obesity and/or type II diabetes at or before disease onset, thus reducing or minimizing the negative effects of obesity and/or type II diabetes. In particular, the present invention allows a better prediction of the risk of obesity and/or type II diabetes and, therefore, of subsequent complications. The methods of the invention can be applied in persons who are free of clinical symptoms and signs of obesity and/or type II diabetes, in those who already have obesity and/or type II diabetes, in those who have family history of obesity and/or type II diabetes, or in those who have elevated level or levels of risk factors of obesity and/or type II diabetes.

In the context of the present invention, a “biomarker” (also herein referred to as a “marker”) is a genetic marker indicative of obesity and/or type II diabetes in humans, that is to say a nucleic acid sequence which is specifically and significantly involved in obesity and/or type II diabetes onset. In the context of the invention, such a marker may also be called an “obesity and/or type II diabetes risk SNP marker” or a “risk SNP marker” or a “risk marker” or a “SNP marker”.

Typically, the genetic markers used in the invention are particular alleles at “polymorphic sites” associated with obesity and/or type II diabetes. A nucleotide position in genome at which more than one sequence is possible in a population is referred to as a “polymorphic site”. Where a polymorphic site is a single nucleotide in length, the site is commonly called an “SNP”. For example, if at a particular chromosomal location, one member of a population has an adenine and another member of the population has a thymine at the same position, then this position is a polymorphic site and, more specifically, the polymorphic site is an SNP. Polymorphic sites may be several nucleotides in length due to, e.g., insertions, deletions, conversions, substitutions, duplications, or translocations. Each version of the sequence with respect to the polymorphic site is referred to as an “allele” of the polymorphic site. Thus, in the previous example, the SNP allows for both an adenine allele and a thymine allele. These alleles are “variant” alleles. Nucleotide sequence variants, either in coding or in non-coding regions, can result in changes in the sequence of the encoded polypeptide, thus affecting the properties thereof (altered activity, altered distribution, altered stability, etc.) Alternatively, nucleotide sequence variants, either in coding or in non-coding regions, can result in changes affecting transcription of a gene or translation of its mRNA. In all cases, the alterations may be qualitative or quantitative or both.

Those skilled in the art will readily recognize that the analysis of the nucleotides present in one or several of the SNP markers disclosed herein in an individual's nucleic acid can be done by any method or technique capable of determining nucleotides present in a polymorphic site. For instance, one may detect biomarkers in the methods of the present invention by performing sequencing, mini-sequencing, hybridisation, restriction fragment analysis, oligonucleotide ligation assay, allele-specific PCR, or a combination thereof. Of course, this list is merely illustrative and in no way limiting. Those skilled in the art may use any appropriate method to achieve such detection.

As it is obvious in the art, the nucleotides present in SNP markers can be determined from either nucleic acid strand or from both strands.

The biomarkers used in the context of the invention are “associated with” the FTO gene, which means that said biomarkers are structurally associated with the FTO gene, e.g., the biomarkers are either in the FTO locus, or in close proximity thereto, and/or that said biomarkers are functionally associated with the FTO gene, e.g., the biomarkers interact with or affect the FTO gene or the expression product thereof.

Preferably, the biomarkers used in the methods and kits of the present invention are selected from the group of single nucleotide polymorphisms (SNPs) listed in anyone of Tables 2, 3, and 6 to 9 below (see part II in the Examples below). Yet preferably, some of the SNPs listed in anyone of Tables 2, 3, and 6 to 9 that are of highly significant predictive value are selected from rs9940128, rs1421085, rs1121980, rs17817449, rs3751812, rs11075990, rs9941349, rs7206790, rs8047395, rs10852521, rs1477196, and rs4783819 . . . . In this group, the SNPs rs9940128, rs1421085, rs1121980, rs3751812, rs7206790, rs8047395, and rs17817449 are of particular interest. Yet more preferably, one will use at least the SNP rs1421085 or rs17817449.

Alternatively, the biomarkers may be polymorphic sites associated with at least one SNP selected from the group listed in anyone of Tables 2, 3, and 6 to 9 below. As defined above, the terms “associated with” mean that said biomarkers are structurally and/or functionally associated with said SNP(s). More specifically, the terms “associated with” mean that said biomarkers are in high linkage disequilibrium with said SNPs, i.e., they present a correlation termed r² of at least 0.6 and/or a D′ of 0.5 with said SNPs in the HapMap European dataset and/or in the population experimentally analyzed by the Inventors as shown below.

Yet alternatively, the biomarkers may be polymorphic sites being in complete linkage disequilibrium with at least one SNP selected from the group listed in anyone of Tables 2, 3, and 6 to 9 below.

Thus, a first aspect of the present invention concerns an in vitro method for risk assessment and/or diagnosis and/or prognosis of obesity and/or type II diabetes in a human subject, comprising at least:

a) detecting, in a nucleic acid sample from said human subject, at least one biomarker associated with the FTO gene; and
b) comparing the biomarker data obtained in step a) from said human subject to biomarker data from healthy and/or diseased people to make risk assessment and/or diagnosis and/or prognosis of obesity and/or type II diabetes in said human subject.

By “risk assessment”, it is meant herein that the present invention makes it possible to estimate or evaluate the risk of a human subject to develop obesity and/or type II diabetes (one could also say “predisposition or susceptibility assessment”). In this respect, an individual “at risk” of obesity and/or type II diabetes is an individual who has at least one at-risk allele or haplotype with one or more “obesity and/or type II diabetes risk SNP markers”. In addition, an “at-risk” individual may also have at least one risk factor known to contribute to the development of obesity and/or type II diabetes, including for instance:
family history of obesity and/or type II diabetes;
some genetic disorders, e.g., Prader-Willi syndrome;
underlying illness (e.g., hypothyroidism);
hypertension and elevated blood pressure;
eating disorders;
certain medications (e.g., atypical antipsychotics);
sedentary lifestyle;
a high glycemic diet, consisting of meals giving high postprandial blood sugar);
weight cycling, caused by repeated attempts to lose weight by dieting;
stressful mentality;
insufficient sleep;
smoking cessation, etc.

The prediction or risk generally implies that the risk is either increased or reduced.

There is no limitation on the type of nucleic acid sample that may be used in the context of the present invention. In this respect, one may use, e.g., a DNA sample, a genomic DNA sample, an RNA sample, a cDNA sample, an hnRNA sample, or an mRNA sample.

The “diseased” people referred to in the methods of the invention are people suffering from obesity and/or type II diabetes.

According to various embodiments, the method described above is useful for:

identifying human subjects at risk for developing obesity and/or type II diabetes;

diagnosing obesity and/or type II diabetes in a human subject;

selecting efficient and safe therapy to a human subject having obesity and/or type II diabetes;

monitoring the effect of a therapy administered to a human subject having obesity and/or type II diabetes;

predicting the effectiveness of a therapy to treat obesity and/or type II diabetes in a human subject in need of such treatment;

selecting efficient and safe preventive therapy to a human subject at risk for developing obesity and/or type II diabetes;

monitoring the effect of a preventive therapy administered to a human subject at risk for developing obesity and/or type II diabetes;

predicting the effectiveness of a therapy to prevent obesity and/or type II diabetes in a human subject at risk.

The terms “treatment” and “therapy” refer not only to ameliorating symptoms associated with obesity and/or type II diabetes, but also preventing or delaying the onset of the disease, and/or also lessening the severity or frequency of symptoms of the disease, and/or also preventing or delaying the occurrence of another episode of the disease.

A second aspect of the present invention relates to an in vitro method for identifying a SNP haplotype associated with obesity and/or type II diabetes susceptibility in a human subject, wherein said method comprises at least:

a) detecting, in a nucleic acid sample from said human subject, at least one SNP of the FTO gene, wherein said at least one SNP is indicative of obesity and/or type II diabetes susceptibility; and
b) identifying said SNP haplotype in said human subject, wherein said SNP haplotype comprises said at least one SNP detected in step a).

As it is well known in the art, a “haplotype” refers to any combination of genetic markers. A haplotype can comprise two or more alleles. The haplotypes (or “at-risk haplotypes”) described herein are found more frequently and significantly in individuals at risk of obesity and/or type II diabetes than in individuals without obesity and/or type II diabetes risk. Therefore, these haplotypes have predictive value for detecting obesity and/or type II diabetes risk, or a susceptibility to obesity and/or type II diabetes in an individual. An “at-risk haplotype” is thus intended to embrace one or a combination of haplotypes described herein over the markers that show high and significant correlation to obesity and/or type II diabetes.

Detecting haplotypes can be accomplished by methods well known in the art for detecting sequences at polymorphic sites.

Preferably, the SNP(s) detected in step a) is(are) selected from the group listed in anyone of Tables 2, 3, and 6 to 9 below.

A third aspect of the present invention provides a test kit for using in an in vitro method to make risk assessment and/or diagnosis and/or prognosis of obesity and/or of type II diabetes in a human subject, wherein said test kit comprises appropriate means for:

a) assessing type and/or level of at least one biomarker associated with the FTO gene in a nucleic acid sample from said human subject; and
b) comparing the biomarker data assessed in a) from said human subject to biomarker data from healthy and/or diseased people to make risk assessment and/or diagnosis and/or prognosis of obesity and/or of type II diabetes in said human subject.

A fourth aspect of the present invention is related to a test kit for using in an in vitro method for identifying a SNP haplotype associated with obesity and/or type II diabetes susceptibility in a human subject, comprising appropriate means for:

a) detecting at least one SNP of the FTO gene in a nucleic acid sample from said human subject, wherein said at least one SNP is indicative of obesity and/or type II diabetes susceptibility; and
b) identifying SNP haplotype in said human subject, wherein said SNP haplotype comprises said at least one SNP detected in a).

The terms “test kit” and “kit” are synonymous and may be used interchangeably.

In the context of the present invention when reference is made to test kits, the terms <<appropriate means>> refer to any technical means useful for achieving the indicated purpose. As non-limiting examples of such appropriate means, one can cite reagents and/or materials and/or protocols and/or instructions and/or software, etc. All the kits of the present invention may comprise appropriate packaging and instructions for use in the methods herein disclosed. The kits may further comprise appropriate buffer(s) and polymerase(s) such as thermostable polymerases, for example Taq polymerase. Such kits may also comprise control primers and/or probes.

According to preferred embodiments, the test kits of the invention may comprise at least:

a) one isolated PCR primer pair consisting of a forward primer and a reverse primer, for specifically amplifying nucleic acids of interest; and/or
b) one isolated primer for specifically extending nucleic acids of interest; and/or
c) one isolated nucleic acid probe specifically binding to nucleic acids of interest; and/or
d) one isolated antibody specifically binding protein( ) encoded by nucleic acid(s) of interest; and/or
e) one microarray or multiwell plate comprising at least one of a) to d) above.

By “nucleic acids of interest”, it is meant herein the nucleic acid regions or segments containing the biomarkers that are indicative of obesity and/or type II diabetes. In this respect, the nucleic acids of interest may be larger than the biomarkers or they may be limited to the biomarkers.

“Probes” and “primers” are oligonucleotides that hybridize in a base-specific manner to a complementary strand of nucleic acid molecules. By “base-specific manner”, it is meant that the two sequences must have a degree of nucleotide complementarity sufficient for the primer or the probe to hybridize. Accordingly, the primer or probe sequence is not required to be perfectly complementary to the sequence of the template. Non-complementary bases or modified bases can be interspersed into the primer or probe, provided that base substitutions do not inhibit hybridization.

A probe or primer usually comprises a region of nucleic acid that hybridizes to at least about 8, preferably about 10, 12, 15, more preferably about 20, 25, 30, 35, and in some cases, about 40, 50, 60, 70 consecutive nucleotides of the nucleic acid template.

The primers and probes are typically at least 70% identical to the contiguous or complementary nucleic acid sequence (which is the “template”). Identity is preferably of at least 80%, 90%, 95%, and more preferably, of 98%, 99%, 99.5%, 99.8%.

Advantageously, the primers and probes further comprise a label, e.g., radioisotope, fluorescent compound, enzyme, or enzyme co-factor.

A fifth aspect of the present invention is directed to a method for selecting pharmaceutical agents useful in prevention and/or treatment of obesity and/or type II diabetes in a human subject, comprising at least:

a) administering the candidate agents to a model living system containing the human FTO gene;
b) determining the effect of said candidate agents on biological mechanisms involving said FTO gene and/or the expression product thereof; and
c) selecting the agents having an altering effect on said biological mechanisms, wherein the selected agents are considered useful in prevention and/or treatment of obesity and/or type II diabetes in a human subject.

By “pharmaceutical agent”, it is referred to either biological agents or chemical agents or both, provided they can be considered as useful in prevention and/or treatment of obesity and/or type II diabetes in a human subject. Examples of biological agents are nucleic acids, including siRNAs; polypeptides, including toxins, enzymes, antibodies, either polyclonal antibodies or monoclonal antibodies; combinations of nucleic acids and polypeptides, and the like. Examples of chemical agents are chemical molecules, chemical molecular complexes, chemical moieties, and the like (e.g., radioisotopes, etc.).

In a sixth aspect, the present invention concerns the use of a model living system containing the human FTO gene for studying pathophysiology and/or molecular mechanisms involved in obesity and/or type II diabetes.

Where reference is made herein to a “model living system”, it is preferably referred to a non-human transgenic animal, or a cultured microbial, insect or mammalian cell, or a mammalian tissue or organ. More preferably, said model living system will express or overexpress the human FTO gene.

A seventh aspect of the present invention relates to an in vitro method for haplotyping the FTO gene in a human subject, comprising at least:

a) detecting, in a nucleic acid sample from said human subject, the nucleotides present at each allelic position of an “obesity and/or type II diabetes susceptibility haplotype”, which haplotype includes at least one of the SNPs listed in anyone of Tables 2, 3, and 6 to 9, or a polymorphism in linkage disequilibrium therewith; and
b) assigning said human subject a particular haplotype according to the nucleotides detected in a).

In a preferred embodiment, this method further comprises the step of determining the risk of said human subject for developing obesity and/or type II diabetes according to the particular haplotype assigned in step b).

The nucleotides present at each allelic position may be detected in step a) of the above method using any appropriate techniques. For instance, this detection may be performed using enzymatic amplification, such as polymerase chain reaction or allele-specific amplification, of said nucleic acid sample. Alternatively, said detection may be done using sequencing.

Besides, the SNPs and haplotypes disclosed herein allow patient stratification. The subgroups of individuals identified as having increased or decreased risk of developing obesity and/or type II diabetes can be used, inter alia, for targeted clinical trial programs and pharmacogenetic therapies wherein knowledge of polymorphisms is used to help identify patients most suited to therapy with particular pharmaceutical agents.

The SNPs and haplotypes described herein represent a valuable information source helping to characterise individuals in terms of, for example, their identity and susceptibility to disease onset/development or susceptibility to treatment with particular drugs.

Therefore, an eighth aspect of the present invention is directed to a method for selecting human subjects for participation in a clinical trial to assess the efficacy of a therapy for treating and/or preventing obesity and/or type II diabetes, comprising at least:

a) grouping the human subjects according to the particular FTO gene haplotype that each human subject belongs to; and
b) selecting at least one human subject from at least one haplotype groups obtained in a) for inclusion in said clinical trial.

In this method, the particular FTO gene haplotype is advantageously determined in vitro by detecting, in a nucleic acid sample from each human subject, the nucleotides present at each allelic position of an “obesity and/or type II diabetes susceptibility haplotype”, which haplotype includes at least one of the SNPs listed in anyone of Tables 2, 3, and 6 to 9, or a polymorphism in linkage disequilibrium therewith.

A ninth aspect of the present invention provides a test kit for in vitro haplotyping the FTO gene in a human subject according to the method as described above, wherein said test kit comprises appropriate means for:

a) detecting, in a nucleic acid sample from said human subject, the nucleotides present at each allelic position of an “obesity and/or type II diabetes susceptibility haplotype”, which haplotype includes at least one SNP selected from the group listed in anyone of Tables 2, 3, and 6 to 9, or a polymorphism in linkage disequilibrium therewith; and
b) assigning said human subject a particular haplotype according to the nucleotides detected in a).

In addition, the present invention concerns, in a tenth aspect, the use of a test kit as described above for stratifying human subjects into particular haplotype groups.

Advantageously, this test kit is further used for selecting at least one human subject from at least one haplotype groups for inclusion in a clinical trial to assess the efficacy of a therapy for treating and/or preventing obesity and/or type II diabetes.

In an eleventh aspect, the present invention is related to a test kit for in vitro determining the identity of at least one SNP selected from the group listed in anyone of Tables 2, 3, and 6 to 9 in the human FTO gene, comprising appropriate means for such determination.

The present invention is illustrated by the non-limiting following figures:

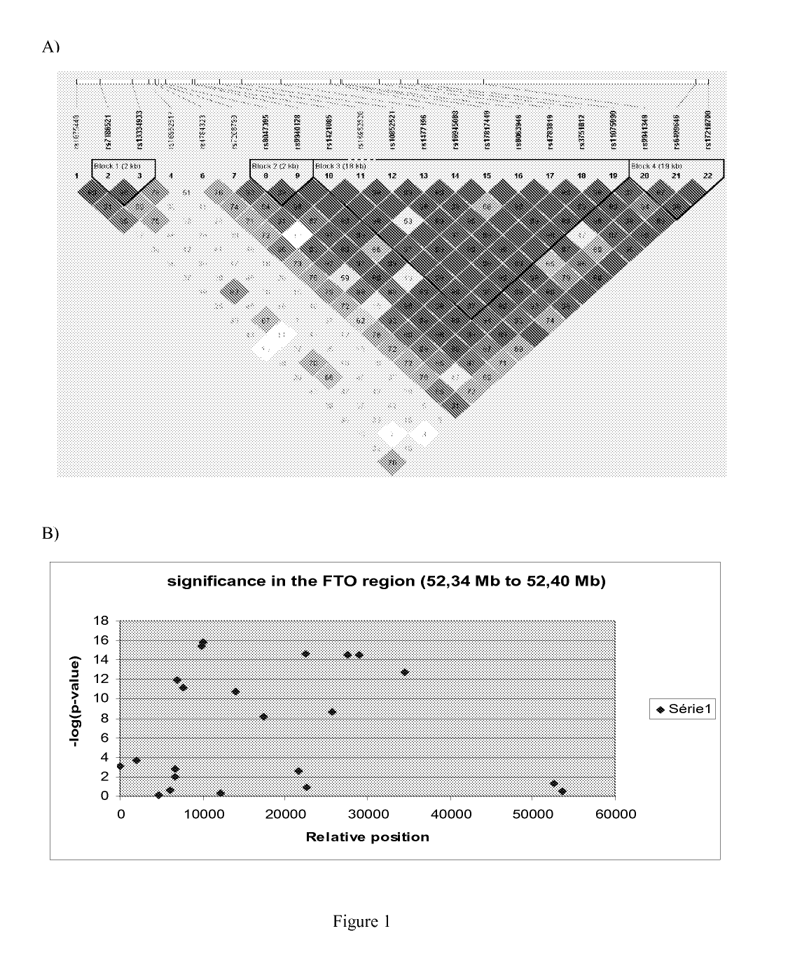

FIG. 1: Linkage disequilibrium structure and association in the FTO region.

A) The linkage disequilibrium is presented as a 2 by 2 matrix where dark grey represents very high linkage disequilibrium (r²) and white absence of correlation between SNPs.

b) For each of the SNPs, the log₁₀ of the p-value for the class III obesity (880 individuals) vs. controls (2700) analysis is shown.

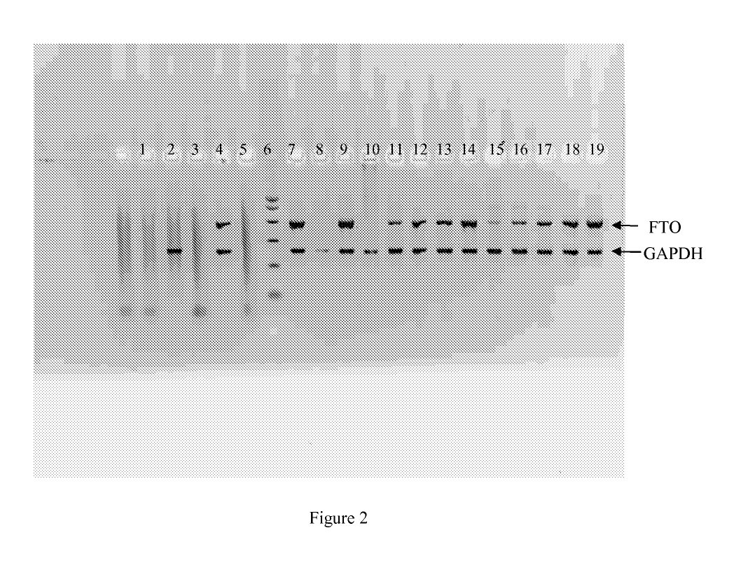

FIG. 2: FTO gene expression in human tissues.

FTO expression in human cDNA from adipose tissue (BioChain Institute, USA), pancreatic islets, FACS-purified beta cells (provided by the Human Pancreatic Cell Core Facility, University Hospital, Lille, France) and multiple tissue cDNA panel (BD Biosciences Clontech) where 1: FTO negative control, 2: GAPDH, 3: GAPDH negative control, 4: GAPDH+FTO, 5: GAPDH+FTO negative control, 6: molecular weight markers 50 bp, 150 bp, 300 bp, 500 bp, 750 bp and 1 kb, 7: adipose tissue, 8: adipose tissue RT minus control, 9: pancreatic islets, 10: pancreatic islets RT minus control, 11: heart, 12: brain, 13: placenta, 14: lung, 15: liver, 16: skeletal muscle, 17: kidney, 18: pancreas, 19: pancreatic beta cells. Glyceraldehyde-3-phosphate dehydrogenase (GAPDH) was used as internal control. Beta cell purity was confirmed by immunochemistry (98% insulin-positive cells) and PCR (absence of amplification with chymotrypsin primers, specific for exocrine cells, and presence of amplification with Pdx1 primers, specific for beta cells). FTO primers used were 5′-TGCCATCCTTGCCTCGCTCA-3′ (SEQ ID No.1) and 5′-TGGGGGCTGAATGGCTCACA-3′ (SEQ ID No.2). These two primers were high-performance liquid chromatography purified. 1 μg of adipose tissue, pancreatic islets and beta cells RNA was randomly reverse transcribed using M-MLV Reverse Transcriptase (Promega, USA) according to instructions. PCR was performed using the FastStart Taq DNA polymerase kit (Roche, Germany) according to instructions with 1.25 mmol/l MgCl₂, 0.4 μmol/l of each primer, and 5 μl single strand cDNA, using the hot-start PCR method modified as follows: 95° C. for 4 min, 40 cycles of 95° C. for 30 s, 68° C. for 2 min, and then 68° C. for 3 min. PCR products were separated on 2% (wt/vol) agarose gel and visualized using ethidium bromide and ultraviolet trans-illumination.

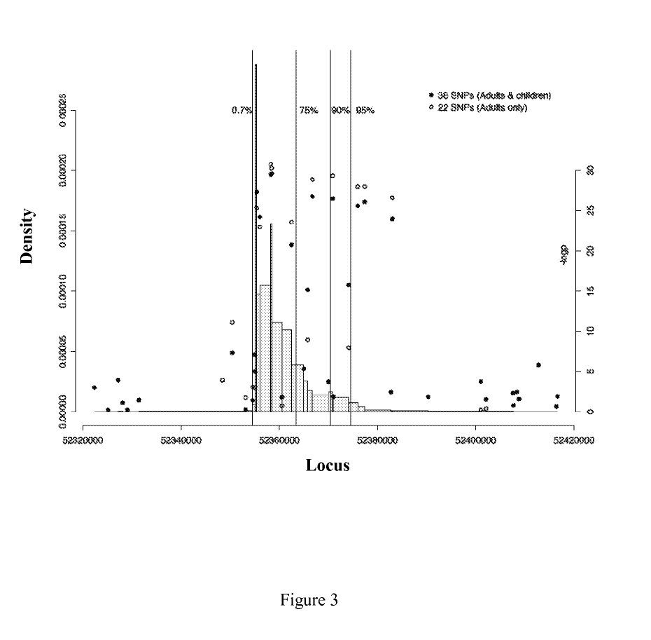

FIG. 3: Distribution of the posterior probability distribution for the location of putative causal locus in the FTO gene. Position is expressed in kb on chromosome 16. Dots represent the log₁₀ of the single SNP association p-value. Lines represent the limits of the 95%, 90% and 75% credible interval.

Other embodiments and advantages of the present invention will be understood upon reading the following Examples.

### EXAMPLES

### I. Materials and Methods

I.1: Statistical analyses

a) Association tests. Logistic regression was used to test association in case-controls under a multiplicative model and Pearson chi-square for the general association model.

The p-values for replication are one-sided for testing the specific hypothesis of increased frequency of allele C (resp. G) in SNPs rs1421085 (resp. rs17817449) in obese children and adults.

Association testing of both SNPs in family based cohorts was performed using the TDT test which compares the number of transmissions of the at-risk allele, from heterozyguous parent to affected offspring, to its expectation. A McNemar X² test assesses the significance.

Fisher's method was used for combining p-values of the different studies, in which the twice the negative sum of the natural log of n p-values follows a X² distribution with 2n degrees of freedom.

b) Genetic model. The proportion of BMI variance explained in adult founders of our familial study populations (parents of French obese children) and in children from the Leipzig cohort, was estimated. The BMI was normalized and expressed in SDS.

The QTL liability threshold model with a quantitative liability trait L (mean 0 and SD 1 in the whole population) and a threshold T, above which an individual is classified as affected, was used. The trait L follows a mixture of three normal distributions N(μ_{g}, σ_{R}). μ_{g} is the genotype specific L mean (takes values −a, 0 and a) and σ²_{R} is the proportion of residual variance which is not due to the locus. For obesity, the trait L can be identified with BMI, as obesity is defined as having a BMI over a certain threshold. With these parameters, it was possible to express the disease risk in terms of Genotype Risk Ratios, GRRᵢ=P(affected/G=i)/P(affected/G=0). The variance due to the locus under investigation was directly derived from the values a and f, the frequency of the at-risk allele in population: σ²_{α}=2.f.(1−f).a² (Eq 1). The percentage of variance explained by the variant (σ²_{α}) was derived from the linear regression model, by inverting Eq 1. Then, the GRRs corresponding to a prevalence of 10%, used for common obesity, was iteratively calculated.

### I.2: Genotyping

Initial case-control genotyping was done by the Applied Biosystems SNPlex™ Technology based on the Oligonucleotide Ligation Assay (OLA) combined with multiplex PCR target amplification (http://vww.appliedbiosystems.com). The chemistry of the assay relies on a set of universal core reagent kits and a set of SNP-specific ligation probes allowing a multiplex genotyping of 48 SNPs simultaneously in a unique sample. A quality control measure was included by using specific internal controls for each step of the assay (according to the manufacturer's instructions). Allelic discrimination was performed through capillary electrophoresis analysis using an Applied Biosystems 3730xl DNA Analyzer and GeneMapper3.7 software. Duplicate samples were assayed with a concordance rate of 100%.

High-throughput genotyping for the variants rs1421085 and rs17817449 in replication samples was performed using the TaqMan® SNP Genotyping Assays (Applied Biosystems, Foster City, Calif. USA). The PCR primers and TaqMan probes were designed by Primer Express and optimized according to the manufacturer's protocol.

All SNPs were in Hardy-Weinberg equilibrium (p>0.05). The call rates were higher than 95% in all and groups of cases and controls from all populations except in Swiss obese individuals.

Call rates and HWE test p-values are displayed in Table 1 below.

TABLE 1
Genotype counts, Hardy Weinberg tests and percentage of successful
genotyping

| | Cases | | | | | Controls | | | | |
|---|---|---|---|---|---|---|---|---|---|---|
| Study | CC (GG) | CT (GT) | TT | pHWE | Missing | CC (GG) | CT (GT) | TT | pHWE | Missi [symbol] |

French population, adult obesity

| | | | | | | | | | |
|---|---|---|---|---|---|---|---|---|---|
| 473 | 1273 | 944 | P = 0.47 | 2.7% | 242 | 425 | 200 | p = 0.88 | 3.2% |
| 439 | 1288 | 948 | P = 0.99 | 3.2% | 235 | 426 | 212 | P = 0.79 | 2.5% |

French population, childhood obesity, study 1

| | | | | | | | | | |
|---|---|---|---|---|---|---|---|---|---|
| 175 | 481 | 323 | P = 0.98 | 3% | 192 | 334 | 173 | p = 0.52 | 4.6% |
| 164 | 489 | 337 | P = 0.84 | 2% | 205 | 320 | 164 | p = 0.2 | 5.6% |

French population, childhood obesity, study 2

| | | | | | | | | | |
|---|---|---|---|---|---|---|---|---|---|
| 95 | 237 | 187 | P = 0.43 | 2.5% | 130 | 233 | 119 | p = 0.77 | 4.7% |
| 92 | 237 | 196 | P = 0.38 | 1.3% | 129 | 236 | 120 | p = 0.84 | 4.1% |

Swiss population, adult obesity

| | | | | | | | | | |
|---|---|---|---|---|---|---|---|---|---|
| 120 | 233 | 161 | P = 0.14 | 5% | 146 | 235 | 123 | p = 0.34 | 9% |
| 109 | 246 | 164 | P = 0.64 | 4% | 135 | 243 | 138 | p = 0.41 | 7% |

German population, childhood obesity

| | | | | | | | | | |
|---|---|---|---|---|---|---|---|---|---|
| 110 | 343 | 246 | P = 0.87 | 1.7% | 79 | 142 | 62 | p = 0.99 | 0.9% |
| 119 | 341 | 231 | P = 0.84 | 2.9% | 81 | 142 | 58 | p = 0.96 | 1.4% |

pHWE: p-value for Hardy Weinberg disequilibrium test.
Missing: percentage of failed genotypes.
Genotype counts for CC, CT and TT (SNP rs1421085), GG, GT and TT (rs17817449) are presented in Table 1 above. [symbol]
indicates data missing or illegible when filed

An unusually high frequency of C (resp. G) allele was observed in controls of the Swiss study which is the control sample with highest missing genotypes rate. This may be due either to presence of undetected obese individuals in this anonymous donors sample or be indicative of a correlation between call rate and allele frequency (differential call rate). However, the samples with the highest call rate and displaying no difference of missing rate between cases and controls (French adult obesity and German children obesity) showed the usual range of allele frequency difference (0.41 to 0.51). Thus, the observed association is unlikely to be due to genotype-dependent calling rate difference in cases and controls.

Besides usual duplicates, 535 obese children and 329 class III obese adults were genotyped both in the case-control and in the familial studies. The concordance rates between these two genotyping techniques were 100% for both SNPs in both studies.

### I.3: Additional Experimental Procedures

### a) Genotypes:

39 SNPs were genotyped in 6833 individuals. They capture 100% of the SNPs with a MAF (Minor Allele Frequency) higher than 1% in a region spanning from position 5234790 kb (rs1861868) to position 52386696 kb (rs13337696).

73% of the individuals (N=5037) were successfully genotyped for the 39 SNPs and 88% (6030 individuals) for at least 38 SNPs. The average call rate was 99%.

### b) Phenotypes:

BMI was calculated and the z-score of BMI was determined according to the Cole's method (Cole et al., 1990).

### c) Statistical Analysis:

Model Selection:

A systematic analysis of all possible combinations of 1 to k polymorphisms to select the most informative and parsimonious haplotype configuration in terms of predicting disease status was performed. Because SNPs are in strong linkage disequilibrium (LD), likelihood was estimated from haplotype analyses for combinations of more than 1 polymorphism. The likelihood generated by the program THESIAS was transformed into a Bayesian Information Criterion (BIC) values for each haplotype model and then subtracted the minimum BIC value obtained for each model over all models explored, giving a rescaled BIC value for each haplotype model. The models with a rescaled BIC-2 are considered equivalent to the most informative model, and among these models, the most parsimonious model with the fewest polymorphisms is considered the best model.

Haplotype Clustering:

HapCluster was used to perform a stochastic search for a case-rich cluster of haplotypes that are similar in the vicinity of a putative risk-enhancing variant. Haplotypes within the cluster are predicted to carry a risk-enhancing allele. The algorithm returns a Bayes factor to summarise the evidence for a causal variant, and a sample from the posterior distribution for its location. The current version, freely available at www.daimi.au.dk/˜mailund/HapCluster/, allows an allelic model, suitable for additive effects, and accepts unphased genotype data. Both these enhancements to the algorithm described in Waldron et al (2006) were employed.

### II. Results

### II.1: Results of Obesity Studies

### II.1.A: First Experimental Results and Examples:

48 SNPs in different intergenic regions were initially selected in order to estimate the distribution of neutral SNPs in French Caucasian case-control obesity data-sets. Surprisingly, the SNP rs1121980, located on chromosome 16q12.2, was found to be strongly associated with severe class III (BMI >40 kg/m²) adult obesity (OR=1.55 [1.39-1.73], p-value=5.3.10⁻¹⁶).

It appeared that this SNP is actually located within the first intron of a newly described gene named fatso or FTO (Peters et al., 1999) that has nine predicted exons in humans and encompasses a large 410,507 bp. genomic region on the NCBI 36.1 human genome assembly. Additional SNPs were tested in a 60-kb region (30 kb on each side of this SNP) which spans the LD block where rs1121980 lies. This region encompasses part of the first intron, second exon and first part of the second intron of the FTO gene. SNPs tagging all the frequent markers (MAF >0.05) with an r² >0.7 as well as SNPs located in potentially functional elements (transcription factor binding sites or other regulatory elements and conserved region between species) and in r² >0.8 with the initial SNP rs1121980, were selected. Twenty-five SNPs were eventually selected, and twenty-three were successfully genotyped. The case control sample comprised 896 class III obese adults (BMI >40 kg m²), and 2,700 non obese French Caucasian controls (BMI <27 kg m²). Both obese adult individuals and controls have been previously described (Meyre et al., 2005).

Results are shown in Table 2 below. Strong association of several SNPs with class III obesity (1.9.10⁻¹⁶≦p≦5.10⁻⁹) was found. Interestingly, three out of the five most significantly associated SNPs, rs17817449, rs3751812 and rs1421085 were putatively functional, based both on phastCons conservation score calculated on 11 vertebrates species (Siepel et al., 2005) and Regulatory Potential score calculated on 7 species (King et al., 2005). Information for genotyped SNPs is displayed in Tables 2 and 3 below.

TABLE 2
Genotype distribution and association tests under the general and the
additive model

| | Status | MAF | N₁₁ (%) | | N₁₂ (%) | | N₂₂ (%) | | general | additive |
|---|---|---|---|---|---|---|---|---|---|---|
| rs1075440 | Non Obese | 0.312 | 1137 | (0.47) | 1026 | (0.43) | 236 | (0.10) | | |
| | Class III | 0.269 | 473 | (0.54) | 330 | (0.38) | 70 | (0.08) | 2,398 · 10⁻⁰³ | 7,877 · 10⁻⁰⁴ |
| rs7186521 | Non Obese | 0.468 | 672 | (0.28) | 1203 | (0.50) | 519 | (0.22) | | |
| | Class III | 0.521 | 203 | (0.23) | 423 | (0.49) | 239 | (0.28) | 5,761 · 10⁻⁰⁴ | 1,670 · 10⁻⁰⁴ |
| rs13334933 | Non Obese | 0.188 | 1584 | (0.66) | 734 | (0.31) | 85 | (0.04) | | |
| | Class III | 0.184 | 582 | (0.66) | 266 | (0.30) | 28 | (0.03) | 8,827 · 10⁻⁰¹ | 6,892 · 10⁻⁰¹ |
| rs16952517 | Non Obese | 0.118 | 1868 | (0.78) | 506 | (0.21) | 31 | (0.01) | | |
| | Class III | 0.126 | 673 | (0.77) | 188 | (0.21) | 17 | (0.02) | 3,731 · 10⁻⁰¹ | 3,594 · 10⁻⁰¹ |
| rs6499643 | Non Obese | 0.159 | 1625 | (0.72) | 573 | (0.25) | 74 | (0.03) | | |
| | Class III | 0.130 | 639 | (0.77) | 175 | (0.21) | 21 | (0.03) | 2,019 · 10⁻⁰² | 5,741 · 10⁻⁰³ |
| rs4784323 | Non Obese | 0.326 | 1080 | (0.45) | 1080 | (0.45) | 243 | (0.10) | | |
| | Class III | 0.283 | 449 | (0.51) | 361 | (0.41) | 68 | (0.08) | 3,618 · 10⁻⁰³ | 7,793 · 10⁻⁰⁴ |
| rs7206790 | Non Obese | 0.524 | 560 | (0.23) | 1176 | (0.49) | 675 | (0.28) | | |
| | Class III | 0.423 | 292 | (0.34) | 407 | (0.47) | 160 | (0.19) | 1,251 · 10⁻¹¹ | 1,439 · 10⁻¹² |
| rs8047395 | Non Obese | 0.481 | 655 | (0.27) | 1187 | (0.49) | 562 | (0.23) | | |
| | Class III | 0.383 | 332 | (0.38) | 413 | (0.47) | 128 | (0.15) | 3,104 · 10⁻¹¹ | 2,626 · 10⁻¹² |
| rs9940128 | Non Obese | 0.426 | 811 | (0.34) | 1155 | (0.48) | 453 | (0.19) | | |
| | Class III | 0.537 | 190 | (0.22) | 420 | (0.49) | 254 | (0.29) | 4,317 · 10⁻¹⁴ | 4,706 · 10⁻¹⁵ |
| rs1421085 | Non Obese | 0.410 | 855 | (0.35) | 1134 | (0.47) | 422 | (0.18) | | |
| | Class III | 0.524 | 200 | (0.23) | 425 | (0.49) | 242 | (0.28) | 7,392 · 10⁻¹⁵ | 7,605 · 10⁻¹⁶ |
| rs16952520 | Non Obese | 0.042 | 2201 | (0.92) | 200 | (0.08) | 2 | (0.00) | | |
| | Class III | 0.038 | 816 | (0.93) | 65 | (0.07) | 1 | (0.00) | 6,535 · 10⁻⁰¹ | 4,131 · 10⁻⁰¹ |
| rs10852521 | Non Obese | 0.479 | 673 | (0.28) | 1171 | (0.48) | 572 | (0.24) | | |
| | Class III | 0.386 | 327 | (0.38) | 415 | (0.48) | 129 | (0.15) | 3,713 · 10⁻¹⁰ | 3,712 · 10⁻¹¹ |
| rs1477196 | Non Obese | 0.368 | 967 | (0.40) | 1112 | (0.46) | 329 | (0.14) | | |
| | Class III | 0.290 | 438 | (0.51) | 340 | (0.40) | 78 | (0.09) | 3,752 · 10⁻⁰⁸ | 5,922 · 10⁻⁰⁹ |
| rs1121980 | Non Obese | 0.429 | 892 | (0.33) | 1270 | (0.48) | 511 | (0.19) | | |
| | Class III | 0.541 | 189 | (0.21) | 436 | (0.49) | 262 | (0.30) | 5,697 · 10⁻¹⁵ | 5,277 · 10⁻¹⁶ |
| rs16945088 | Non Obese | 0.089 | 2001 | (0.83) | 394 | (0.16) | 17 | (0.01) | | |
| | Class III | 0.067 | 750 | (0.87) | 109 | (0.13) | 3 | (0.00) | 1,666 · 10⁻⁰² | 3,493 · 10⁻⁰³ |
| rs17817449 | Non Obese | 0.402 | 860 | (0.36) | 1152 | (0.48) | 388 | (0.16) | | |
| | Class III | 0.513 | 212 | (0.24) | 426 | (0.49) | 235 | (0.27) | 7,554 · 10⁻¹⁵ | 1,442 · 10⁻¹⁵ |
| rs8063946 | Non Obese | 0.057 | 2150 | (0.89) | 258 | (0.11) | 8 | (0.00) | | |
| | Class III | 0.048 | 782 | (0.91) | 80 | (0.09) | 1 | (0.00) | 2,886 · 10⁻⁰¹ | 1,416 · 10⁻⁰¹ |
| rs4783819 | Non Obese | 0.371 | 943 | (0.39) | 1122 | (0.47) | 325 | (0.14) | | |
| | Class III | 0.289 | 443 | (0.52) | 337 | (0.39) | 80 | (0.09) | 2,780 · 10⁻⁰⁹ | 8,596 · 10⁻¹⁰ |
| rs3751812 | Non Obese | 0.399 | 885 | (0.37) | 1141 | (0.47) | 394 | (0.16) | | |
| | Class III | 0.505 | 218 | (0.25) | 425 | (0.49) | 226 | (0.26) | 3,038 · 10⁻¹³ | 4,121 · 10⁻¹⁴ |
| rs11075990 | Non Obese | 0.401 | 871 | (0.36) | 1146 | (0.48) | 394 | (0.16) | | |
| | Class III | 0.509 | 211 | (0.25) | 421 | (0.49) | 227 | (0.26) | 1,037 · 10⁻¹³ | 1,483 · 10⁻¹⁴ |
| rs9941349 | Non Obese | 0.412 | 843 | (0.35) | 1154 | (0.48) | 417 | (0.17) | | |
| | Class III | 0.513 | 211 | (0.24) | 422 | (0.49) | 233 | (0.27) | 4,986 · 10⁻¹² | 7,420 · 10⁻¹³ |
| rs6499646 | Non Obese | 0.096 | 1965 | (0.81) | 434 | (0.18) | 16 | (0.01) | | |
| | Class III | 0.079 | 747 | (0.85) | 123 | (0.14) | 8 | (0.01) | 2,238 · 10⁻⁰² | 2,797 · 10⁻⁰² |
| rs17218700 | Non Obese | 0.120 | 1859 | (0.77) | 518 | (0.22) | 31 | (0.01) | | |
| | Class III | 0.111 | 688 | (0.80) | 159 | (0.18) | 16 | (0.02) | 8,785 · 10⁻⁰² | 2,774 · 10⁻⁰¹ |

MAF: the minor allele frequency.
N₁₁, N₁₂ and N₂₂ are the genotype frequencies for the frequent allele homozygote, the heterozygote and the rare allele homozygote, respectively.
General: result of the general test model test, a Pearson χ² test with 2 degrees of freedom comparing the genotype frequencies in case and control.
Additive: result of the logistic regression of the case-control status on the number of at-risk alleles.

TABLE 3
Assessment of SNP's putative functionality

| position | rs | genomatix | conservation | Regulatory Potential |
|---|---|---|---|---|
| 52357007 | rs9937053 | | 0 | — |
| 52357405 | rs9928094 | | 0.000708661 | 0 |
| 52357477 | rs9930333 | | 0.00283465 | 0 |
| 52358068 | rs9939973 | | 0.00283465 | 0 |
| 52358129 | rs9940646 | | 0 | 0.0695239 |
| 52358254 | rs9940128 | | 0 | 0.24681 |
| 52358454 | rs1421085 | | 1 | 0.31981 |
| 52359049 | rs9923147 | | 0.0136693 | 0.0697798 |
| 52359485 | rs9923544 | | 0 | 0 |
| 52361074 | rs1558902 | | 0 | 0.0609172 |
| 52362707 | rs11075985 | | 0 | 0 |
| 52366747 | rs1121980 | | 0 | 0 |
| 52368186 | rs7193144 | | 0 | 0 |
| 52370867 | rs17817449 | * | 0.992126 | 0.286477 |
| 52370950 | rs8043757 | | 0.0393701 | 0.0860575 |
| 52373775 | rs8050136 | | 0.304016 | 0.198246 |
| 52374252 | rs8051591 | | 0 | 0 |
| 52374338 | rs9935401 | | 0.00179528 | 0 |
| 52375960 | rs3751812 | * | 1 | 0.326163 |
| 52376669 | rs9936385 | | 0.0393701 | 0.029236 |
| 52376698 | rs9923233 | | 0 | 0.0498732 |
| 52377377 | rs11075989 | | 0 | — |
| 52377393 | rs11075990 | | 0 | — |
| 52379362 | rs7201850 | | 0.0060315 | 0 |
| 52380151 | rs7185735 | | 0 | 0 |
| 52382988 | rs9941349 | | 0.76378 | 0 |
| 52384679 | rs9931494 | | 0 | 0.00920513 |
| 52385566 | rs17817964 | | 0.661748 | 0.158021 |
| 52387952 | rs9930501 | | 0 | 0 |
| 52387965 | rs9930506 | | 0 | 0 |
| 52387991 | rs9932754 | | 0.0530236 | 0 |

For each SNP, it is reported in Table 3 above the physical position in bp using NCBI assembly Build 35, the phastCons conservation score calculated on 11 vertebrates species and Regulatory Potential score calculated on 7 species. A star is added when the SNP inserts or deletes a Transcription Factor Binding Site (using SNP inspector Tool from Genomatix Suite). In bold are indicated the three SNPs having the highest scores and then being most likely functional.

It was also tested whether the association observed in the whole region was reflecting one unique signal or whether any other SNP or haplotype displays association on its own, and concluded that the at-risk alleles were nearly perfect proxies of each other. Thus, at least these three SNPs are likely to mirror one unique association of a haplotype combining derived alleles (from NCBI) with a frequency of 40% in controls.

As recently outlined (Ott, 2004), the replication of association data in additional samples is necessary to exclude spurious conclusions, especially when the pre study odd for the implication of a gene is low, which is the case for fatso. SNPs rs1421085 and rs17817449 were chosen, because they display very high evidence of association and are putatively functional, to carry out these analyses. All the p-values were one-sided in these analyses.

It was first compared allele frequencies of the selected SNPs in 1,010 non obese French individuals (Hercberg et al. 1998) (SUVIMAX cohort, BMI <27 kg m²) with 736 obese children (mean age=11 y, BMI >97^{th} percentile) and found significant association with early onset obesity (OR=1.28 [1.11-1.47] p=2.10⁻⁵ and OR=1.25 [1.09-1.44] p=5.10⁻⁴ for rs1421085 and rs17817449, respectively). Then, 532 non obese young French adults (Vu-Hong et al., 2006) (Haguenau cohort, median age=21y, BMI <25 kg/m²) and 505 French obese children with a BMI >97^{th} percentile (Le Fur et al., 2002) from Saint Vincent de Paul Hospital, were analyzed. Again, similar trend for association with early onset obesity was found (OR=1.47 [1.23-1.75], p=1.17.10⁻⁵ and OR=1.52 [1.28-1.81], p=1.82.10⁻⁶ for rs1421085 and rs17817449, respectively). Finally, 700 lean children (mean age=11.7y, BMI between 16^{th} and 85^{th} percentile) and 283 obese children (mean age=11.7y, BMI >90^{th} percentile), both of German Caucasian origin (Korner et al., 2007), were genotyped.

Association was again confirmed for both SNPs (OR=1.69 [1.38-2.06], p=3.46.10⁻⁷, and OR=1.65 [1.35-2.01], p=1.23.10⁻⁶ for rs1421085 and rs17817449, respectively). Table 4 below shows the effect size estimation.

TABLE 4
Effect size estimation for rs1421085

| | m ZBMI | m Age | a | σ²ₐ | GRR₁ | PAR |
|---|---|---|---|---|---|---|
| French | 1.02 | 55 y | 0.19 [0.09-0.28] | 0.017 [0.004-0.038] | 1.41 [1.17-1.69] | 0.27 [0.13-0.40] |
| | SDS | | | | | |
| German | 0.46 | 11.7 y | 0.12 [0.03-0.20] | 0.007 [0.0005-0.019] | 1.24 [1.05-1.44] | 0.18 [0.04-0.29] |
| | SDS | | | | | |
| | | | | | 1.31 [1.16-1.48] | 0.22 [0.12-0.31] |

m ZBMI: mean of the BMI expressed in standard deviations
m Age: mean age in the study population
a: additive effect, estimated by the slope of the regression of ZBMI on the number of at-risk alleles
σ²ₐ: genetic variance (it is here assumed no deviation from the additive model). As the whole variance is 1, the genetic variance is equivalent to heritability
GRR₁: Genotype Risk Ratio between penetrance of wild type homozygote and heterozygote
PAR: population attributable risk

557 Swiss class III obese adults and 541 anonymous Swiss donors were also genotyped, and it was further replicated the initial association between fatso and obesity (OR=1.26 [1.07-1.49], p=0.0032 and OR=1.21 [1.02-1.43] p=0.01 for rs1421085 and rs17817449, respectively). Of note, although allele frequencies in Swiss obese subjects were consistent with the initial observations in French obese subjects (MAF=0.50), the Swiss blood donor cohort which was not tested for obesity displayed higher allele frequencies (f=0.46 vs. 0.41), which may be explained by the presence of obesity in this anonymous individuals group.

For each status, overall significance was assessed using the Fisher's method which combines p-values of each independent analysis. The number of effective tests (Nyholt, 2004) was used at each step, 16.72 and 1.2 respectively, to correct for multiple testing while accounting for the between SNPs' correlation. The meta-analysis combining evidence of association for obesity gave very significant results: p-value=1.67.10⁻²⁶ and p=1.07.10⁻²⁴ for SNP rs1421085 and rs17817449, respectively. In order to exclude a potential undetected stratification effect, these 2 SNPs were genotyped in the parents and sibs of both French obese children and class III obese adults. An over-transmission of the SNP rs1421085 (rs17817449 respectively) obesity “at risk” C (respectively G) allele to both obese children and adults was observed (% transmitted=57%, p-value=1.10⁻⁴ and % transmitted=66%, p-value=0.00045 in obese children for rs1421085 and rs17817449, respectively; % transmitted=57%, p-value=2.5.10⁻⁴ and % transmitted=62%, p-value=0.005, in obese adults for rs1421085 and rs17817449, respectively). An additional cohort comprising 154 families, discordant for severe obesity, (with at least one class III obese and one lean sib) of Swedish descent was further analyzed, and it was also observed over-transmission of the same allele to obese offspring (% transmitted=61%, p-value=0.05 for both SNPs). The overall significance of these three combined family based studies is 2.8.10⁻⁶.

Moreover, in founders of French Childhood Obesity families dataset, it was found a very strong association with BMI corrected for age and sex for both SNPs (β=0.19 [0.09-0.29], p=8.10⁻⁵ and β=0.17 [0.07-0.27], p=4.10⁻⁴ for rs1421085 and rs17817449, respectively). All replication results are displayed in Table 5 below and genotype counts are shown in Table 1 above.

TABLE 5
Analyses and effect estimates in the study populations

| | | | | | | |
|---|---|---|---|---|---|---|
| Study | SNP | Genotyped | Obese | Controls | f case/con | Multiplicative model |

A/Independent Adult case-control obesity studies

| | | | | | | |
|---|---|---|---|---|---|---|
| Initial Adult obesity | rs1421085 | 3278 | 867 | 2411 | 0.52/0.41 | OR = 1.56 [1.40-1.75] p = 7.6 · 10⁻¹⁶ |
| | rs17817449 | 3273 | 873 | 2400 | 0.51/0.40 | OR = 1.56 [1.40-1.75] p = 1.44 · 10⁻¹⁵ |
| Swiss Adult obesity | rs1421085 | 1018 | 504 | 514 | 0.52/0.46 | OR = 1.26 [1.07-1.49] p = 0.0032* |
| | rs17817449 | 1035 | 516 | 519 | 0.50/0.47 | OR = 1.21 [1.02-1.43] p = 0.01* |

| | | |
|---|---|---|
| Fisher test statistic: −2 * Σln(p-values) | rs1421085 | p = 3.54 · 10⁻¹⁵ |
| Fisher test statistic: −2 * Σln(p-values) | rs17817449 | p = 1.99 · 10⁻¹⁴ |

B/Independent Case-control studies on Childhood obesity

| | | | | | | |
|---|---|---|---|---|---|---|
| French Childhood | rs1421085 | 1681 | 702 | 979 | 0.48, 0.41 | OR = 1.28 [1.11-1.47] p = 2 · 10⁻⁵* |
| obesity 1 | rs17817449 | 1683 | 693 | 990 | 0.47, 0.40 | OR = 1.25 [1.09-1.44] p = 5 · 10⁻⁴* |
| French Childhood | rs1421085 | 1001 | 482 | 519 | 0.51, 0.40 | OR = 1.47 [1.23-1.75] p = 1.17 · 10⁻⁵* |
| Obesity 2 | rs17817449 | 1010 | 485 | 525 | 0.51, 0.40 | OR = 1.52 [1.28-1.81] p = 1.82 · 10⁻⁶* |
| German Childhood | rs1421085 | 982 | 283 | 699 | 0.53, 0.40 | OR = 1.69 [1.38-2.06] p = 3.46 · 10⁻⁷* |
| Obesity | rs17817449 | 972 | 281 | 691 | 0.54, 0.42 | OR = 1.65 [1.35-2.01] p = 1.23 · 10⁻⁶* |

| | | |
|---|---|---|
| Fisher test statistic: −2 * Σln(p-values) | rs1421085 | p = 9.8 · 10⁻¹⁴ |
| Fisher test statistic: −2 * Σln(p-values) | rs17817449 | p = 1.7 · 10⁻¹² |

C/Overall significance of the case-control studies

| | | |
|---|---|---|
| Fisher test statistic: −2 * Σln(p-values) | rs1421085 | p = 1.67 · 10⁻²⁶ |
| Fisher test statistic: −2 * Σln(p-values) | rs17817449 | p = 1.07 · 10⁻²⁴ |

D/Family-based studies

| | | Informative | | | | |
|---|---|---|---|---|---|---|
| Study | | Meïoses | Tr | Non Tr | Tr./Non Tr. | Allelic Model |
| French Childhood | rs1421085 | 685 | 392 | 293 | 1.37 | p = 1 · 10⁻⁴* |
| Obesity | rs17817449 | 707 | 401 | 306 | 1.31 | p = 2.5 · 10⁻⁴* |
| French Adult obesity | rs1421085 | 81 | 73 | 38 | 1.9 | p = 0.00045* |
| | rs17817449 | 81 | 70 | 43 | 1.6 | p = 0.005* |
| Swedish Adult obesity | rs1421085 | 54 | 33 | 21 | 1.57 | p = 0.05* |
| | rs17817449 | 47 | 29 | 18 | 1.61 | p = 0.05* |

| | |
|---|---|
| Fisher test statistic: −2 * Σln(p-values) | p = 2.8 · 10⁻⁶ |

The results of case-control and family-based analyses are shown in sections A, B and D of Table 5 above, for each cohort. Association analyses compared genotype frequencies in obese and non obese individuals using logistic regression. The OR is the risk increase according to the number of at-risk alleles.
In section C, the significance of the meta-analysis combining all case-controls studies, adults and children is shown. Each p-value is corrected by the number of effective tests inferred from the LD matrix before being added into the Fisher test statistic.
Section D shows the number of transmitted (Tr.) and un-transmitted (Non Tr.) alleles in the three familial samples.
*All the p-values, except those of the initial samples are one-sided.
** Includes trios of grand-parents and parents of the initial childhood obesity study.

### II.1.B: Additional Experimental Results and Examples:

Using haplotype clustering methods (Molitor et al., 2003), a fine-mapping analysis was performed to restrict the localization of the underlying causal variant. 39 SNPs, spanning 100 kb which include the 47 kb as well adjacent blocks were genotyped in 6933 individuals, including 2446 controls and 1935 obese adults and children (Table 6 below). This design covers, with r² >0.8, all the HapMapSNPs displaying a MAF higher than 1% in this region.

TABLE 6

| SNP | A1 | F obese | F lean | A2 | χ² | P | OR | Lower-Upper |
|---|---|---|---|---|---|---|---|---|
| rs4280233 | 4 | 0.04473 | 0.05002 | 3 | 1.242 | 0.2652 | 0.8894 | [0.7236-1.093] |
| rs9925311 | 1 | 0.03215 | 0.02949 | 3 | 0.4973 | 0.4807 | 1.093 | [0.853-1.402] |
| rs6499640 | 3 | 0.3662 | 0.3895 | 1 | 4.688 | 0.03038 | 0.9058 | [0.8282-0.9907] |
| rs16952479 | 4 | 0.06127 | 0.06012 | 1 | 0.04812 | 0.8264 | 1.02 | [0.8519-1.222] |
| rs16952482 | 2 | 0.1072 | 0.1107 | 4 | 0.2608 | 0.6096 | 0.9645 | [0.8394-1.108] |
| rs6499641 | 4 | 0.4766 | 0.4975 | 1 | 3.411 | 0.06477 | 0.9199 | [0.8419-1.005] |
| rs9933611 | 3 | 0.02262 | 0.02391 | 1 | 0.1498 | 0.6987 | 0.9447 | [0.7084-1.26] |
| rs7186521 | 3 | 0.5145 | 0.4787 | 1 | 10.52 | 0.001182 | 1.154 | [1.058-1.258] |
| rs13334933 | 3 | 0.1873 | 0.1825 | 1 | 0.307 | 0.5795 | 1.032 | [0.9234-1.153] |
| rs16952517 | 1 | 0.1309 | 0.1194 | 3 | 2.524 | 0.1122 | 1.111 | [0.9757-1.265] |
| rs6499643 | 2 | 0.1256 | 0.1583 | 4 | 16.73 | 4.303e−05 | 0.7638 | [0.6711-0.8693] |
| rs4784323 | 1 | 0.295 | 0.3192 | 3 | 5.604 | 0.01792 | 0.8926 | [0.8125-0.9807] |
| rs7206790 | 2 | 0.4327 | 0.5139 | 3 | 54.24 | 1.778e−13 | 0.7217 | [0.6616-0.7873] |
| rs8047395 | 3 | 0.3979 | 0.4725 | 1 | 46.28 | 1.026e−11 | 0.7379 | [0.6759-0.8055] |
| rs9940128 | 1 | 0.5247 | 0.4385 | 3 | 61.41 | 4.642e−15 | 1.414 | [1.296-1.542] |
| rs1421085 | 2 | 0.5115 | 0.4231 | 4 | 64.99 | 7.541e−16 | 1.427 | [1.309-1.557] |
| rs16952520 | 3 | 0.03309 | 0.03853 | 1 | 1.724 | 0.1892 | 0.854 | [0.6745-1.081] |
| rs10852521 | 4 | 0.3997 | 0.4717 | 2 | 43.24 | 4.847e−11 | 0.7456 | [0.6831-0.8139] |
| rs16952522 | 3 | 0.05676 | 0.04565 | 2 | 5.423 | 0.01988 | 1.258 | [1.037-1.526] |
| rs1477196 | 1 | 0.305 | 0.3592 | 3 | 26.65 | 2.44e−07 | 0.7829 | [0.7134-0.8592] |
| rs1121980 | 1 | 0.5273 | 0.4402 | 3 | 56.7 | 5.072e−14 | 1.418 | [1.295-1.554] |
| rs16945088 | 3 | 0.06847 | 0.08512 | 1 | 7.766 | 0.005323 | 0.79 | [0.6691-0.9327] |
| rs17817449 | 3 | 0.4964 | 0.4134 | 4 | 57.52 | 3.343e−14 | 1.399 | [1.282-1.526] |
| rs8063946 | 4 | 0.04396 | 0.05283 | 2 | 3.413 | 0.06467 | 0.8244 | [0.6716-1.012] |
| rs4783819 | 3 | 0.3067 | 0.3621 | 2 | 27.87 | 1.3e−07 | 0.7792 | [0.7102-0.8549] |
| rs3751812 | 4 | 0.4938 | 0.4106 | 3 | 57.91 | 2.74e−14 | 1.4 | [1.284-1.527] |
| rs11075990 | 3 | 0.4964 | 0.4127 | 1 | 58.51 | 2.027e−14 | 1.403 | [1.286-1.53] |
| rs9931164 | 3 | 0.008224 | 0.01356 | 1 | 5 | 0.02535 | 0.6034 | [0.3857-0.9439] |
| rs9941349 | 4 | 0.5023 | 0.4241 | 2 | 51.02 | 9.16e−13 | 1.371 | [1.257-1.495] |
| rs2111650 | 3 | 0.01647 | 0.01841 | 1 | 0.4422 | 0.5061 | 0.8929 | [0.6394-1.247] |
| rs6499646 | 2 | 0.0774 | 0.08874 | 4 | 3.389 | 0.06564 | 0.8616 | [0.7351-1.01] |
| rs17218700 | 1 | 0.111 | 0.1232 | 3 | 2.929 | 0.08703 | 0.8884 | [0.7757-1.017] |
| rs11075994 | 1 | 0.274 | 0.3014 | 3 | 7.372 | 0.006623 | 0.8748 | [0.7943-0.9635] |
| rs1421090 | 2 | 0.2742 | 0.2698 | 4 | 0.2103 | 0.6466 | 1.023 | [0.9284-1.127] |
| rs9939811 | 2 | 0.2649 | 0.2552 | 4 | 1.017 | 0.3133 | 1.052 | [0.9534-1.16] |
| rs9972717 | 1 | 0.1818 | 0.1807 | 3 | 0.01635 | 0.8982 | 1.007 | [0.9005-1.127] |
| rs11075995 | 1 | 0.2441 | 0.2268 | 4 | 3.395 | 0.06538 | 1.101 | [0.9939-1.22] |
| rs11075997 | 4 | 0.4549 | 0.4481 | 2 | 0.3921 | 0.5312 | 1.028 | [0.9426-1.121] |
| rs7195539 | 3 | 0.03607 | 0.04031 | 1 | 0.9829 | 0.3215 | 0.8909 | [0.7088-1.12] |

The distribution of posterior location probability (FIG. 3), obtained using the HapCluster program (Waldron et al., 2006), highlights the SNPs rs7206790, rs8047395, rs9940128, rs1421085, which also individually display very significant evidence of association (2.10-12-5.10-16, Table 6 above). The 95% credible interval is 20 kb long (chr16:52354480-52374503) while the 90% and the 75% credible interval reduce the interesting region down to 16 kb (chr16:52354480-52370450) and 9 kb (chr16:52354480-52363464), respectively (FIG. 3).

Actually, it appears that all the markers in the interval chr16:52344480-5240000 which are in high LD r² >0.7 with rs1421085 in European populations are of interest in the context of the present invention (Table 7).

TABLE 7
List of SNPs identified in Hap Map

rs1421085
rs1558902
rs7193144
rs7185735
rs17817964
rs9937053
rs8043757
rs8050136
rs9935401
rs3751812
rs9939609
rs12149832
rs8051591
rs11075990
rs17817449
rs11075989
rs9923233
rs9940128
rs9923147
rs9923544
rs1121980
rs9928094
rs9939973
rs9941349
rs9930333
rs11075985
rs9940646
rs9931494
rs11642841
rs9936385
rs7201850
rs9930506
rs9922708
rs9930501
rs9932754
rs9922619
rs17817288
rs8057044
rs8055197
rs1861866
rs10852521
rs9922047
rs8047395
rs8044769
rs11075987

Thus, in spite of a very high LD in this region, significant difference in association with obesity status was found along this region. The posterior probability distribution is in agreement with the fine-scale recombination data as retrieved from HapMap (www.hapmap.org) (FIG. 3.).

96 individuals have been sequenced in the 20 kb region. This permitted to identify 66 new SNPs (not yet reported in dbSNP for “Single Nucleotide Polymorphism database”), which are set forth in Table 8A hereunder according to their position. These SNPs were not identified so far at least because the number of individuals used for the human genome sequence assembly is not large enough to ensure statistical power to detect all frequent genetic variations. Using 96 individuals gave here for the first time sufficient power to discover frequent SNPs (MAF >0.05). 62 dbSNPs validated through the above described re-sequencing procedure are listed in Table 8B below. 26 dbSNPs not found through the above described re-sequencing procedure are listed in Table 8C below.

Because this is the largest sequencing study performed so far in this region (96 individuals), the Inventors were both able to identify new SNPs (i.e., not listed in dbSNP nor in HapMap) and to confirm (or discard) previously identified SNPs (either in dbSNP, HapMap or in any other public database). It is noteworthy that all the SNPs (confirmed and new) are in the scope of the present invention as they are in strong linkage disequilibrium (high r² and/or D′) with the defined at-risk SNPs (including rs1421085).

TABLE 8A
List of SNPs identified by sequencing (identified by their
position in NCBI 36)

| SNP # | rs# (dbSNP) | MAF | pos. NCBI 36.1 |
|---|---|---|---|
| SNPneg4 | none | 0.194736842 | 52353168 |
| SNPneg3 | none | 0.005263158 | 52353634 |
| SNPneg2 | none | 0.005263158 | 52353765 |
| SNPneg1 | none | 0.410526316 | 52354389 |
| SNP1 | none | 0.115789474 | 52354669 |
| SNP2 | none | 0.089473684 | 52354683 |
| SNP3 | none | 0.005263158 | 52354785 |
| SNP4 | none | 0.089473684 | 52354952 |
| SNP9 | none | 0.005376344 | 52355481 |
| SNP10 | none | 0.02688172 | 52355646 |
| SNP13 | none | 0.068421053 | 52356074 |
| SNP16 | none | 0.094736842 | 52356142 |
| SNP17 | none | 0.005263158 | 52356154 |
| SNP18 | none | 0.005555556 | 52356744 |
| SNP19 | none | 0.477777778 | 52356772 |
| SNP20 | none | 0.477777778 | 52356779 |
| SNP21 | none | 0.477777778 | 52356779 |
| SNP24 | none | 0.005263158 | 52357344 |
| SNP31 | none | 0.015789474 | 52357886 |
| SNP34 | none | 0.049450549 | 52358079 |
| SNP35 | none | 0.049450549 | 52358081 |
| SNP37 | none | 0.005494505 | 52358134 |
| SNP40 | none | 0.484210526 | 52358842 |
| SNP41 | none | 0.010526316 | 52358949 |
| SNP44 | none | 0.005263158 | 52359216 |
| SNP47 | none | 0.010526316 | 52360019 |
| SNP48 | none | 0.010638298 | 52360259 |
| SNP52 | none | 0.484210526 | 52360723 |
| SNP53 | none | 0.010526316 | 52360915 |
| SNP56 | none | 0.005263158 | 52361963 |
| SNP61 | none | 0.015789474 | 52363026 |
| SNP62 | none | 0.010526316 | 52363102 |
| SNP63 | none | 0.047368421 | 52363330 |
| SNP66 | none | 0.484210526 | 52363953 |
| SNP67 | none | 0.005263158 | 52364046 |
| SNP68 | none | 0.026315789 | 52364496 |
| SNP69 | none | 0.110526316 | 52364505 |
| SNP70 | none | 0.005263158 | 52364632 |
| SNP73 | none | | 52365511 |
| SNP75 | none | 0.021052632 | 52365927 |
| SNP77 | none | 0.484210526 | 52366623 |
| SNP79 | none | 0.005263158 | 52367338 |
| SNP80 | none | 0.068421053 | 52367361 |
| SNP81 | none | 0.015789474 | 52367580 |
| SNP82 | none | 0.005263158 | 52367812 |
| SNP84 | none | 0.026315789 | 52368135 |
| SNP86 | none | 0.389473684 | 52368443 |
| SNP87 | none | 0.010526316 | 52368871 |
| SNP88 | none | 0.005263158 | 52369038 |
| SNP89 | none | 0.352631579 | 52369288 |
| SNP90 | none | 0.005263158 | 52369652 |
| SNP91 | none | 0.026315789 | 52369843 |
| SNP92 | none | 0.021052632 | 52369917 |
| SNP96 | none | 0.026315789 | 52370646 |
| SNP101 | none | 0.021052632 | 52371452 |
| SNP102 | none | 0.005263158 | 52371611 |
| SNP104 | none | 0.005263158 | 52371763 |
| SNP105 | none | 0.021052632 | 52371788 |
| SNP108 | none | 0.236842105 | 52371970 |
| SNP109 | none | 0.005263158 | 52372172 |
| SNP110 | none | 0.015789474 | 52372327 |
| SNP111 | none | 0.005263158 | 52372419 |
| SNP114 | none | 0.1 | 52373229 |
| SNP115 | none | 0.021276596 | 52373248 |
| SNP117 | none | 0.005319149 | 52373665 |
| SNP123 | none | 0.126315789 | 52374818 |

TABLE 8B
List of validated dbSNPs

| rs# | | pos. NCBI |
|---|---|---|
| (dbSNP) | MAF | 36.1 |
| rs13334933 | 0.2 | 52353136 |
| rs16952517 | 0.067 | 52354557 |
| rs6499642 | 0.021052632 | 52355006 |
| rs6499643 | 0.157894737 | 52355018 |
| rs4784323 | 0.278947368 | 52355065 |
| rs7206790 | 0.430107527 | 52355408 |
| rs28429148 | 0.446236559 | 52355819 |
| rs8047395 | 0.378947368 | 52356023 |
| rs8049424 | 0.012195122 | 52356113 |
| rs8047587 | 0.408536585 | 52356122 |
| rs9937521 | 0.477777778 | 52356796 |
| rs28562191 | 0.477777778 | 52356803 |
| rs9937354 | 0.484210526 | 52357347 |
| rs9928094 | 0.484210526 | 52357405 |
| rs9930333 | 0.484210526 | 52357477 |
| rs9930397 | 0.484210526 | 52357485 |
| rs9940278 | 0.484210526 | 52357700 |
| rs9932600 | 0.263157895 | 52357772 |
| rs12446228 | 0.284210526 | 52357887 |
| rs9939973 | 0.484210526 | 52358068 |
| rs9940646 | 0.483516484 | 52358129 |
| rs9940128 | 0.483516484 | 52358254 |
| rs1421085 | 0.472527473 | 52358454 |
| rs35418808 | 0.021052632 | 52358996 |
| rs9923147 | 0.484210526 | 52359049 |
| rs9923544 | 0.484210526 | 52359485 |
| rs11642015 | 0.478947368 | 52359994 |
| rs16952520 | 0.052631579 | 52360538 |
| rs8055197 | 0.373684211 | 52360656 |
| rs1558901 | 0.473684211 | 52360687 |
| rs1558902 | 0.484210526 | 52361074 |
| rs1861866 | 0.373684211 | 52361840 |
| rs10852521 | 0.373684211 | 52362465 |
| rs12447107 | 0.042105263 | 52362592 |
| rs11075985 | 0.473684211 | 52362707 |
| rs11075986 | 0.1 | 52362844 |
| rs2058908 | 0.184210526 | 52363645 |
| rs9922047 | 0.373684211 | 52363780 |
| rs16952522 | 0.168421053 | 52364998 |
| rs17817288 | 0.415789474 | 52365264 |
| rs1477196 | | 52365758 |
| rs16952523 | 0.026315789 | 52366194 |
| rs1121980 | 0.468421053 | 52366747 |
| rs7187250 | 0.389473684 | 52368046 |
| rs7193144 | 0.389473684 | 52368186 |
| rs8063057 | 0.389473684 | 52369933 |
| rs16945088 | 0.073684211 | 52370024 |
| rs8057044 | 0.463157895 | 52370114 |
| rs17817449 | 0.388297872 | 52370867 |
| rs8043757 | 0.389473684 | 52370950 |
| rs8063946 | 0.052631579 | 52370998 |
| rs28623715 | 0.005263158 | 52371760 |
| rs28500763 | 0.005263158 | 52371818 |
| rs9972653 | 0.384210526 | 52371863 |
| rs11075987 | 0.484210526 | 52372661 |
| rs17817497 | 0.352631579 | 52372935 |
| rs8054237 | 0.037234043 | 52373365 |
| rs8050136 | 0.384210526 | 52373775 |
| rs4783819 | 0.273684211 | 52374147 |
| rs8051591 | 0.389473684 | 52374252 |
| rs4783820 | 0.026315789 | 52374284 |
| rs9935401 | 0.389473684 | 52374338 |

TABLE 8C
List of dbSNPs not found by re-sequencing

| rs# | pos. NCBI |
|---|---|
| (dbSNP) | 36.1 |
| rs34467788 | 52353146 |
| rs13336126 | 52353565 |
| rs28595108 | 52353664 |
| rs35186040 | 52354012 |
| rs28525169 | 52355433 |
| rs17217467 | 52355624 |
| rs4784324 | 52355886 |
| rs12929439 | 52356770 |
| rs11383210 | 52356984 |
| rs28715938 | 52357937 |
| rs28690649 | 52358303 |
| rs1421086 | 52358843 |
| rs35592467 | 52358958 |
| rs13335913 | 52359213 |
| rs7190757 | 52363517 |
| rs35744826 | 52363745 |
| rs5816907 | 52364505 |
| rs10718688 | 52364506 |
| rs9924817 | 52365424 |
| rs9927087 | 52365752 |
| rs16952524 | 52366484 |
| rs35938047 | 52366800 |
| rs16952525 | 52367514 |
| rs34256655 | 52368017 |
| rs34621076 | 52368532 |
| rs10614742 | 52373008 |

### II.2: Results of Type II Diabetes Studies

Table 9 below shows the results of case control analysis on 2400 controls (part of the controls used in the obesity studies described above) and 2200 type II diabetes patients of French Caucasian origin. Analysis was performed under the additive model.

TABLE 9
Association with Type II diabetes in the FTO region

| SNP name | p-value additive |
|---|---|
| rs1075440 | 0.000509958 |
| rs7186521 | 0.000594697 |
| rs13334933 | 0.604536 |
| rs16952517 | 0.00221782 |
| rs6499643 | 0.245951 |
| rs4784323 | 0.207249 |
| rs7206790 | 5.67355 · 10⁻⁵ |
| rs8047395 | 0.000100524 |
| rs9940128 | 2.37984 · 10⁻⁶ |
| rs1421085 | 8.47986 · 10⁻⁶ |
| rs16952520 | 0.0216816 |
| rs10852521 | 0.00112251 |
| rs1477196 | 0.00301224 |
| rs1121980 | 3.89511 · 10⁻⁶ |
| rs16945088 | 0.0296665 |
| rs17817449 | 2.74261 · 10⁻⁵ |
| rs8063946 | 0.109686 |
| rs4783819 | 0.00446752 |
| rs3751812 | 6.25013 · 10⁻⁵ |
| rs11075990 | 2.35022 · 10⁻⁵ |
| rs9941349 | 5.16178 · 10⁻⁵ |
| rs6499646 | 0.00346673 |
| rs17218700 | 0.816384 |

### III. Conclusions

Fatso (FTO) function is mostly unknown. Mice heterozygous for an FTO syntenic Fused toes (Ft) are characterized by partial syndactyly of forelimbs and massive thymic hyperplasia indicating that programmed cell death is affected. Homozygous Ft/Ft embryos die at mid-gestation and show severe malformations of craniofacial structures. However, this physical inactivation involves several genes in the region and thus these phenotypes are not necessarily related to FTO itself. In humans, a small chromosomal duplication has been identified on large chromosomal 16q12.2 region which includes the fatso (FTO) locus (Stratakis et al., 2000). Besides mental retardation, dysmorphic facies, and digital anomalies, the authors also report obesity as primary symptom. Fatso (FTO) locus variation was also recently reported to be modestly associated with the metabolic syndrome in French Canadian hypertensive families (Seda et al., 2005).

FTO's gene expression was examined in several human tissues, especially those of interest for obesity such as brain, adipose tissue, and it was found that human fatso gene was expressed in all eleven tested tissues as shown in FIG. 2. In addition, the microarray based Gene Expression Database of the Novartis Research Foundation's Genomics Institute (“GNF”/SymAtlas) indicates that fatso is highly expressed in human hypothalamus, pituitary and adrenal glands suggesting a potential role in the hypothalamic-pituitary-adrenal axis (HPA) implicated in body weight regulation (Su et al., 2004) (http://symatlas.gnf.org/SymAtias/). Moreover, the protein has no identified structural domain (Peters et al., 1999) and no informed network link to any other proteins (Ingenuity software tools) which could help to predict its function and its physiological role.

Here, it is shown that several potentially functional SNPs in fatso (FTO) locus are highly associated with early onset and severe obesity in European population. The calculated Population Attributable Risk of 0.22, which is explained by the high frequency of the at-risk haplotype, argues for a putative important effect on population corpulence. It appears to be the most significant association reported so far for obesity (Lyon et al., 2007). Also, it is shown here that the same SNPs are highly associated with type II diabetes.

It was recently shown that, although most research findings in genetic studies may be accidental, multiple replication of strong associations greatly enhances the positive predictive value of research findings being true, even if the pre study odd is low (Moonesinghe et al., 2007). In this regard, fatso appears to be a gene with a strong contribution to obesity as well as type II diabetes despite its, yet, unknown role in glucose homeostasis.

### REFERENCES

1. Peters, T., Ausmeier, K. & Ruther, U. Cloning of Fatso (Fto), a novel gene deleted by the Fused toes (Ft) mouse mutation. Mamm Genome 10, 983-6 (1999).
2. Meyre, D. et al. Variants of ENPP1 are associated with childhood and adult obesity and increase the risk of glucose intolerance and type 2 diabetes. Nat Genet 37, 863-7 (2005).
3. Siepel, A. et al. Evolutionarily conserved elements in vertebrate, insect, worm, and yeast genomes. Genome Res 15, 1034-50 (2005).
4. King, D. C. et al. Evaluation of regulatory potential and conservation scores for detecting cis-regulatory modules in aligned mammalian genome sequences. Genome Res 15, 1051-60 (2005).
5. Ott, J. Association of genetic loci: Replication or not, that is the question. Neurology 63, 955-8 (2004).
6. Hercberg, S. et al. A primary prevention trial using nutritional doses of antioxidant vitamins and minerals in cardiovascular diseases and cancers in a general population: the SU.VI.MAX study—design, methods, and participant characteristics. SUpplementation en Vltamines et Mineraux AntioXydants. Control Clin Trials 19, 336-51 (1998).
7. Vu-Hong, T. A. et al. The INS VNTR locus does not associate with smallness for gestational age (SGA) but interacts with SGA to increase insulin resistance in young adults. J Clin Endocrinol Metab 91, 2437-40 (2006).
8. Le Fur, S., Le Stunff, C. & Bougneres, P. Increased Insulin Resistance in Obese Children Who Have Both 972 IRS-1 and 1057 IRS-2 Polymorphisms. Diabetes 51, S304-307 (2002).
9. Korner, A., Berndt, J., Stumvoll, M., Kiess, W. & Kovacs, P. TCF7L2-gene polymorphisms confer an increased risk for early impairment of glucose metabolism and increased height in obese children. J Clin Endocrinol Metab (2007).
10. Nyholt, D. R. A simple correction for multiple testing for single-nucleotide polymorphisms in linkage disequilibrium with each other. Am J Hum Genet 74, 765-9 (2004).
11. Stratakis, C. A. et al. Anisomastia associated with interstitial duplication of chromosome 16, mental retardation, obesity, dysmorphic facies, and digital anomalies: molecular mapping of a new syndrome by fluorescent in situ hybridization and microsatellites to 16q13 (D16S419-D16S503). J Clin Endocrinol Metab 85, 3396-401 (2000).
12. Ŝeda O, T. J., Merlo E, Gaudet D, Broeckel U, Bouchard G, Antoniol G, Brunelle P-L, Gurau A, Gossard F, Pintos J, Kotchen T A, Cowley A W, Hamet P. The Genomic Signatures of Hypertension. Hypertension 46, 875-887 (2005).
13. Lyon, H. N. et al. The association of a SNP upstream of INSIG2 with Body Mass Index is reproduced in several but not all cohorts. PLoS Genetics preprint, e61.eor (2007).
14. Moonesinghe, R., Khoury, M. J. & Janssens, A. C. Most published research findings are false-but a little replication goes a long way. PLoS Med 4, e28 (2007).
15. Su et al. Proc Nat Acad Sci USA 101, 6062-7 (2004).
16. Molitor, J., Marjoram, P. & Thomas, D. Fine-scale mapping of disease genes with multiple mutations via spatial clustering techniques. Am J Hum Genet 73, 1368-84 (2003).
17. Waldron, E. R., Whittaker, J. C. & Balding, D. J. Fine mapping of disease genes via haplotype clustering. Genet Epidemiol 30, 170-9 (2006).
18. Cole, T. J., Freeman, J. V. & Preece, M. A. Body mass index reference curves for the UK, 1990. Arch Dis Child 73, 25-9 (1995).
19. McVean, G. A. et al. The fine-scale structure of recombination rate variation in the human genome. Science 304, 581-4 (2004).
20. Hudson, R. R. Two-locus sampling distributions and their application. Genetics 159, 1805-17 (2001).

1. An in vitro method for risk assessment and/or diagnosis and/or prognosis of obesity and/or type II diabetes in a human subject, comprising:
a) detecting, in a nucleic acid sample from said human subject, at least one biomarker associated with FTO gene; and
b) comparing biomarker data obtained in step a) from said human subject to biomarker data from healthy and/or diseased people to make risk assessment and/or diagnosis and/or prognosis of obesity and/or type II diabetes in said human subject.

2. The method according to claim 1, wherein said at least one biomarker is selected from the group consisting of single nucleotide polymorphisms (SNPs) listed in anyone of Tables 2, 3, and 6 to 9.

3. The method according to claim 1, wherein said at least one biomarker is a polymorphic site associated with at least one SNP selected from the group consisting of SNPs listed in anyone of Tables 2, 3, and 6 to 9.

4. The method according to claim 1, wherein said at least one biomarker is a polymorphic site being in complete linkage disequilibrium with at least one SNP selected from the group consisting of SNPs listed in anyone of Tables 2, 3, and 6 to 9.

5. The method according to claim 1, wherein said method is for identifying human subjects at risk for developing obesity and/or type II diabetes.

6. The method according to claim 1, wherein said method is for diagnosing obesity and/or type II diabetes in a human subject.

7. The method according to claim 1, wherein said method is for selecting efficient and safe therapy to a human subject having obesity and/or type II diabetes.

8. The method according to claim 1, wherein said method is for monitoring the effect of a therapy administered to a human subject having obesity and/or type II diabetes.

9. The method according to claim 1, wherein said method is for predicting the effectiveness of a therapy to treat obesity and/or type II diabetes in a human subject in need of such treatment.

10. The method according to claim 1, wherein said method is for selecting efficient and safe preventive therapy to a human subject at risk for developing obesity and/or type II diabetes.

11. The method according to claim 1, wherein said method is for monitoring the effect of a preventive therapy administered to a human subject at risk for developing obesity and/or type II diabetes.

12. The method according to claim 1, wherein said method is for predicting the effectiveness of a therapy to prevent obesity and/or type II diabetes in a human subject at risk.

13. The method according to claim 1, wherein said at least one biomarker is rs9940128, rs1421085, rs1121980, rs17817449, rs3751812, rs11075990, rs9941349, rs7206790, rs8047395, rs10852521, rs1477196, or rs4783819.

14. The method according to claim 13, wherein said at least one biomarker is rs9940128, rs1421085, rs1121980, rs3751812, rs7206790, rs8047395, or rs17817449.

15. A test kit for using in an in vitro method according to claim 1, comprising appropriate means for:
a) assessing type and/or level of at least one biomarker associated with the FTO gene in a nucleic acid sample from a human subject; and
b) comparing the biomarker data assessed in a) from said human subject to biomarker data from healthy and/or diseased people to make risk assessment and/or diagnosis and/or prognosis of obesity and/or of type II diabetes in said human subject.


SEQUENCE LISTING (2 sequences)

SEQ ID NO: 1 - DNA, 20 bp, Artificial sequence
  Fto primer
tgccatccttgcctcgctca

SEQ ID NO: 2 - DNA, 20 bp, Artificial sequence
  Fto primer
tgggggctgaatggctcaca